(19) United States
(12) Patent Application Publication
(10) Pub. No.: US 2026/0232339 A1
(43) Pub. Date: Aug. 13, 2026

(54) LASER-ASSISTED ASPIRATION THROMBECTOMY

(71) Applicant: UNIVERSITY OF SOUTH FLORIDA, TAMPA, FL (US)

(72) Inventors: Mitchell Robert Harrah, Tampa, FL (US); Maxim V. Mokin, Tampa, FL (US); Ashwin Bharadwaj Parthasarathy, Tampa, FL (US)

(21) Appl. No.: 19/539,603

(22) Filed: Feb. 13, 2026

Related U.S. Application Data
(60) Provisional application No. 63/758,034, filed on Feb. 13, 2025.

(51) Int. Cl. A61B 17/221; A61B 17/22; A61B 18/24; A61B 18/26; B23K 26/0622

(52) U.S. Cl. CPC A61B 17/221; A61B 17/22; A61B 18/245; A61B 18/26; B23K 26/0624

(57) ABSTRACT

A combination of aspiration and configured laser ablation procedures for target removal. In a specific case, embodiments of proposed methodology are utilized as aspiration thrombectomy assisted with the use of a laser source judiciously chosen to operate in a pulsed regime with pulse durations exceeding a nanosecond at a wavelength that is determined based on a figure-of-merit that representing both a stress confinement within a material of the target and absorption of the laser radiation by this material.

### CROSS-REFERENCE TO RELATED APPLICATIONS

This patent application claims the benefit of, and the priority from U.S. Provisional Application No. 63/758,034 filed on Feb. 13, 2025. The entire contents of which are hereby incorporated by reference herein.

### RELATED ART

A thrombus is the coalescence of blood platelets and fibrin in blood vessels, which can cause disruptions in blood flow substantially in any blood vessel and ischemic injury in tissue. Thrombi are known to be heterogeneous in composition, ranging from highly composed of red blood cells to highly composed of fibrin. Ultimately, the composition of the thrombus can alter the structure and properties of the clotted tissue, such as fibrin-heavy clots being tightly bound (see, for example, Staessens, S. and De Meyer, S. F, Thrombus heterogeneity in ischemic stroke. Platelets 32, 331-339; 2021).

The development of a thrombus can have sweeping effects within the body—in particular those related to large vessel occlusions. In the peripheral vasculature, thrombi can disrupt flow to cause conditions like Deep Vein Thrombosis (DVT). Deep vein thrombosis (DVT) is a growing public health issue within the United States (Skeik, N. et al., in J. Vasc. Surg. Venous Lymphat. Disord. 9, 1062-1070.e6 2021; Brækkan, S. K. et al., in J. Thromb. Haemost. 14, 1978-1987; 2016). DVT is responsible for increasing hospital expenses by about $13 billion annually.

Currently, most thrombi are treated with what is referred to as antithrombolytics or with mechanical thrombectomy procedures. Antithrombolytics are a pharmacological approach to break apart the thrombus (Blann, A. D., Landray, M. J. & Lip, G. Y. H., in BMJ 325, 762; 2002). However, the effectiveness of these therapies has decreased with the rise of risk factors like obesity, and anti-thrombolytics may need to be administered within a short window of thrombi formation for them to be most effective.

Mechanical thrombectomy methods, including special retrievable devices (such as stent retrievers) and aspiration catheters, have been established as a low-resource methodology for thrombus removal (Sweid et al., in Stroke and Vascular Neurology, 5, e000262, 2019) and remain the current standard of care for treatment of large vessel occlusions. The relative compositions of the three components of thrombi (red blood cells (RBCs), platelets, and fibrin) largely dictates the possible outcomes of a mechanical thrombectomy. The efficacy of mechanical thrombectomy methods such as stent retrievers depends on the size and composition of the thrombi. For example, RBC rich thrombi are fragile and can be dislodged with mechanical force. Such secondary thrombi can occlude more distal vessels not reachable with traditional mechanical thrombectomy devices, leading to additional tissue injury. With stent retrievers, the thrombus is encased in a net-like device and is removed by pulling it out of the patient (Jolugbo, P., & Ariens, R. A. S., in Stroke, 52(3), 1131-1142, 2021). With aspiration, the thrombus undergoes a negative pressure (suction) to be removed through a vacuum/suction catheter. For severe blockages, endovascular therapy is the preferred method to remove thrombi and either used in conjunction with antithrombolysis or alone. Mechanical thrombectomy (aspiration) alone is known to be often unsuccessful for cases with large clot burden, and, as is known in related art, aspiration thrombectomy has lower success rates with thrombi with higher fibrin concentrations. Further, aspiration thrombectomy leads to post-thrombotic syndrome rates of approximately 28.1% after one year from the surgery, where thrombi particles that have not been completely removed cause downstream blockages. The so-called laser thrombolysis has found limited use in clinical settings—in fact, prior studies have focused on the mechanisms of laser thrombolysis (see, for example, Zhang, H. et al., in J. Biophotonics 15, e202200197, 2022), the effects of continuous and long pulse width lasing (see, for example, Viator, J. A. & Prahl, S. A. in Lasers Surg. Med. 25, 379-388, 1999), and on efficacy measurements (see, for example, den Heijer, P. et al., in J. Intervent. Cardiol. 7, 525-534, 1994; Aludin, S. et al., in Cardiovasc. Intervent. Radiol. 45, 228-235, 2022). Notably, in most of the related art studies Nd:YAG, helium:YAG, and excimer lasers were utilized, all of which are expensive and have significant space utilization and utilities consumption considerations. Arguably, these costs make this technology unaffordable for many clinics that have smaller budgets.

### SUMMARY OF THE INVENTION

As the skilled artisan will readily appreciate—and while the examples of embodiment and applications of embodiments of the proposed methodology are described in specific reference to a diode-laser assisted aspiration thrombectomy—the proposed methodology can generally be implemented to and applied to substantially removal or dislodging of substantially any target or object, be such object an animate and/or biological object or an inanimate object. AS such the discussed below implementations remain but specific example(s) and the scope of the invention includes the methodology addressing a target of the general nature.

Embodiments of the invention provide an apparatus for removal of an identified target. The apparatus includes an aspiration system (that contains a tubular body and a dedicated piping network operably cooperated with the tubular body and that is structured to generate suction at an end of the tubular body), an optical system containing a laser (which laser is configured to operate in a pulsed regime to generate pulses of laser radiation having durations substantially longer than a nanosecond at a wavelength that is defined at least in part by a spectral dependency of a figure of merit representing both a stress confinement within a material of the target and absorption of the laser radiation by the material of the target), and an optical waveguide judiciously located to deliver the laser radiation to the end of the tubular body. At least in one specific implementation, the laser includes a laser diode device. Optionally—but substantially in every implementation—the apparatus may contain electronic circuitry electrically coupled with the aspiration system and/or the optical system. Such electronic circuitry is configured at least to govern an operation of an aspiration system driver (to reversibly produce the suction) and to operate the optical system (to reversibly or switchably produce the pulses of laser radiation) and/or to vary a pulse width, a pulse duty cycle, and an average power of the laser radiation; and/or to vary a strength of the suction produced by the aspiration system. In some implementations, the electronic circuitry (which may be a programmable processor) may be used to determine, based at least on pre-assessed characteristics of the material of the target, the spectral dependency of the FOM and to produce indicia representing the choice of operational wavelength of the laser. Additionally or in the alternative—and substantially in every implementation—the apparatus may additionally contain a sheath dimensioned to slidingly fit into a blood vessel and to removably fit therein both the tubular body and the optical fiber; and/or the tubular body may be dimensioned to removably fit the optical waveguide in the hollow thereof.

Embodiments of the invention additionally provide a method for removal of a target, which may—in a specific non-limiting case—employ substantially every embodiment of the apparatus alluded to above. The method includes at least the following steps: determining the spectral dependency of the FOM within a pre-defined spectral range based at least on pre-assessed material properties of the target; identifying the wavelength of operation of the laser in the apparatus based at least on the spectral dependency of the FOM; operating the laser to generate pulses of the laser radiation substantially at the wavelength of operation; and substantially simultaneously with or in alternating fashion with said operating the laser, generating the suction at the end of the tubular body with the aspiration system. Optionally, and substantially in every implementation of the method, the step of identifying may include determining a value of a wavelength-dependent product of a stress confinement factor in the material of the target and a value of a wavelength-dependent coefficient of absorption of laser radiation by the material of the target. At least one implementation of the method may include a step of channeling the laser radiation within the optical waveguide and spatially converging, outside of the tubular body (and, in a specific case, onto the target). Here, the laser radiation is being delivered along the tubular body to the end of the tubular body. Alternatively or in addition—and substantially in every implementation—the method may additionally include the steps of positioning the end of the tubular body and an output end of the optical waveguide in a vicinity of the target (the target may be affixed to or located at a reference surface); applying the laser radiation to the target to dislodge the target and/or to chip away a portion of the target from a body thereof; and repositioning the target with respect to the reference surface and/or the portion of the target with respect to the body of the target with the suction. In at least some specific cases, the target may include a biological tissue inside a bodily cavity and, in a very specific case, a thrombus within a blood vessel (substantially in every part of the body).

Embodiments further include a computer program product for use on a computer system operating an aspiration thrombectomy apparatus. The computer program product includes a computer usable tangible non-transitory storage medium having computer readable program code thereon. The computer readable program code contains program code for one or more of: (1) assessing a range of operational wavelengths of a laser source (of an embodiment of the above-identified apparatus, which laser source is configured to assist in operation of the apparatus) based at least in part on determining a spectral dependency of a figure of merit that represents both a stress confinement within a material of the target thrombus and absorption of the laser radiation in the material of the target thrombus; (2) governing an operation of the laser source to generate laser radiation at a wavelength within the range of operational wavelengths in a pulsed regime with a pulse duration substantially longer than a nanosecond; (3) switching off and on an aspiration system of the apparatus to reversibly generate suction at an identified end of a suction catheter of the aspiration system to which the laser radiation has been delivered; (4) varying a pulse width, a duty cycle, and/or an average power of the laser radiation; (5) varying a strength of the suction generated by the apparatus. Here, the operating of the aspiration thrombectomy apparatus may include producing the suction substantially simultaneously with generating the laser radiation or producing the suction in alternating fashion with the generating the laser radiation.

### BRIEF DESCRIPTION OF THE DRAWINGS

The invention will be more fully understood by referring to the following Detailed Description of Specific Embodiments in conjunction with the Drawings, of which:

FIG. 1 is a schematic of the embodiment of the laser-assisted aspiration apparatus structured according to the idea of the invention. Some of conventional components or devices that may be required for operation of the apparatus (such as a pump or a valve, or a spectrophotometer) are not necessarily shown.

FIG. 2 illustrates the spectral dependency of a figure of merit devised to represent the efficiency of exposure of the target biological tissue to the multi-factor stress produced by the assisting ablation process (in this example—with laser pulses at 100 μs duration). The graph displays the wavelength dependence towards the mechanical effects within a simulated thrombus. A mark has been made at about the 405 nm to display one of the preferred wavelengths for aspiration-assisting laser radiation.

FIGS. 3A and 3B provide additional details of the schematic of an embodiment of the experimental setup 100 employed to effectuate a specific example of the laser thrombolysis procedure (FIG. 3A) (that employs phantoms of thrombi) on a benchtop sample chamber and a flow diagram of the procedure detailing the measurement and lasing process (FIG. 3B) on a benchtop sample chamber.

FIG. 4: absorbance of 100% ECF thrombi analogs at 500 μs pulse widths with varying duty cycles.

FIG. 5: absorbance of 100% ECF thrombi analogs at 1000 μs pulse widths with varying duty cycles.

FIG. 6: absorbance of 20% ECF thrombi analogs at 500 μs pulse widths with varying duty cycles.

FIG. 7: absorbance of 20% ECF thrombi analogs at 1000 μs pulse widths with varying duty cycles.

### DETAILED DESCRIPTION

As current methods of chemical (using a medication/drug) thrombolysis and mechanical thrombectomy are often not beneficial or adequate, the idea of the present invention stems from the realization that a combination of the aspiration thrombectomy and laser thrombolysis (and, specifically, the laser-assisted aspiration thrombectomy) provides for an affordable and effective removal of thrombi. In that, it is the reinforcement of the aspiration thrombectomy with the judicious use of the low-cost laser thrombolysis—necessarily implemented with the use of a laser apparatus that does not require ultra-short (for example, femtosecond) light pulses. Accordingly, an embodiment of the apparatus of the present invention is thus devoid of a source of light producing ultra-short pulses—that was unexpectedly proven to be of particular advantage. In short, in stark contradistinction to the systems of related art—implementations of the aspiration thrombectomy methodology with the assistance of the laser ablation, according to the idea of the invention, employ an off-the-shelf high-powered laser diode operating at substantially longer (as compared to the currently used for the purposes of the laser ablation excimer lasers) pulse durations of nano-seconds and greater (as discussed in the specific example—a millisecond long pulses), and a custom optical fiber used to deliver the laser light output, produced by such laser diode source, to the target. The skilled artisan will readily appreciate that the assistance to the aspiration of the target occluding a given channel (in a specific example—a target such as a thrombus within a blood vessel) provided by the pulsed laser irradiation, turns on chipping away portions of the target simultaneously or alternatingly with aspiration (i.e., suction to remove) of the chipped away portions of the target and/or on employing the laser ablation to loosen the target within the given channel to dislodge the target for aspiration (i.e., suction to remove). The unexpected advantages provided by such multi-tool methodology include the freedom and ability to use substantially longer optical fibers (as compared to those currently employed in apparatus configured for laser thrombolysis) due to the operationally insignificant (if present at all) dispersion of longer laser light pulses used in operation of an embodiment of the invention upon propagation of such pulses through the optical fiber. Moreover, the proposed longer than nanosecond-pulse-duration laser assisted aspiration thombectomy not only has higher efficiency (as compared with the sole use of aspiration) but, understandably, causes lower blood loss and shorter thrombus removal times.

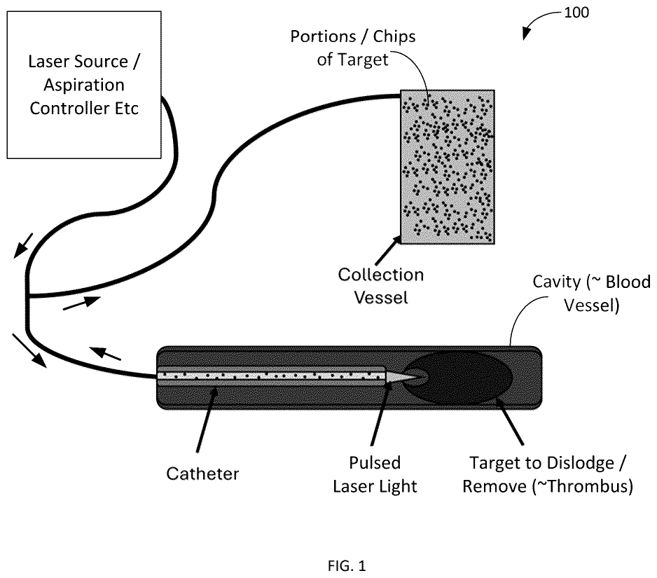

In reference to the schematic of FIG. 1, the implementation of the laser-assisted aspiration thrombectomy apparatus 100 contains an aspiration mechanism (including the aspiration controller) and system of piping connecting such controller with the aspiration catheter (which, in operation of the apparatus, is delivered towards and into the cavity of channel containing the target) that is configured to remove the debris and particulates towards and into the dedicated debris chamber (shown as a collection vessel), and a laser thrombolysis source of radiation and optical fiber that carries the pulsed laser light towards the same target through or in parallel to the aspiration catheter, (for example), both of which will be enclosed in an outer diameter shell or housing (not shown).

With laser thrombolysis portion of the catheter—the laser light with the appropriately identified operational characteristics (pulse width, duty cycle, power) will successfully ablate the thrombi of all compositions. Notably, unlike a pure mechanical thrombectomy procedure, laser ablation does not require any extra force for removal of tougher, high-fibrin content thrombi. This reduces risk of free-thrombi generation. Laser light can be delivered within a standard aspiration catheter and would not require larger bores. By design, small thrombi particulates generated by laser ablation will manifest only at the opening of the aspiration catheter—which will be promptly aspirated out by the negative pressure. This is an important step to avoid any downstream occlusions and reduces embolism risk.

Below, the efficacy of the embodiment of the proposed apparatus to ablate numerous thrombi analogs of various platelet and fibrin concentrations is demonstrated, evidencing the adequacy of the proposed methodology in ablating fibrin-heavy thrombi through long-pulsed laser ablation. Contents of each reference or publication identified in this disclosure are incorporated herein by reference.

### Operating Principle

The idea of the invention stems from the realization that the ablation of target thrombi in laser thrombolysis (used as an assisting tool to the aspiration thrombolytics) can be effectuated by utilizing much longer—as compare with related art—and, specifically, pulses of light having widths (durations) generally longer than a nanosecond. Although ablation could arguably occur under continuous wave irradiation, the skilled artisan knows well that in such a case the ablative effects are vastly ineffective and could cause thermal damage to the surrounding vessel walls: see, for example, Crea, F., Fenech, A., Smith, W., Conti, C. R. & Abela, G. S. in J. Am. Coll. Cardiol. 6, 1052-1056, 1985).

To assess the target ablation conditions under the pulsed irradiation, the thermal relaxation time and the mechanical relaxation time must be calculated to find the pulse length thresholds to contain the thermal and acoustic energy, respectively. The thermal relaxation time, tₜ, is calculated as

$${t}_{t} = \frac{1}{κ ( 3 {μ}_{a} ( {μ}_{a} + {μ}_{s}^{′} ) )} .$$

### Here μₐ and

$${μ}_{s}^{′}$$

are the absorption and reduced scattering coefficients of the target tissue (in this example—thrombus), and κ is the thermal diffusivity given by

$$κ = \frac{K}{{C}_{p} ρ} ,$$

where K is the thermal conductivity, Cₚ is the specific heat at constant pressure and ρ is the density of the target tissue (see Vogel, A. & Venugopalan, V. in Chem. Rev. 103, 577-644, 2003).

To find the mechanical impact of the aspiration-assisting laser ablation process, the mechanical stress (σₚ) generated by laser ablation is considered according to

$${σ}_{p} = A {Γμ}_{a} {Φ}_{0} ,$$

where A is the stress confinement factor, a measure of how efficient the stress generated by the laser pulse is confined within the material, Γ is Grüneisen coefficient, Φ₀ is the incident radiant exposure. The stress confinement factor (A) further depends on the laser pulse width (tₚ) and the mechanical relaxation time (tₘ) and can be calculated as

$$A = \frac{1 - {e}^{- \frac{{t}_{p}}{{t}_{m}}}}{\frac{{t}_{p}}{{t}_{m}}} ,$$

with tₘ=δ/v, where v is the speed of sound in the material. The mechanical relaxation time is a measure of the dispersion of mechanical energy in the material. δ is the light penetration depth given by

$$δ = \sqrt{\frac{1}{3 {μ}_{a} ( {μ}_{a} + {μ}_{s}^{′} ) .}}$$

where μₐ, is the absorption coefficient of the material of the target (here—thrombus),

$${μ}_{s}^{′}$$

is the reduced scattering coefficient given by

$${μ}_{s}^{′} = {μ}_{s} ( 1 - g ) , {μ}_{s}^{′}$$

is the scattering coefficient, and g is the anisotropy of the material of the target. Notably, it is important to recognize that each of μₐ,

$${μ}_{s}^{′}$$

(and hence δ and A) characteristics is wavelength-dependent and thus the spectral behavior and efficiency of the laser ablation assisting process is not trivial and should be judiciously considered to devise the proper procedure.

According to the idea of the invention, the figure of merit—the product Aμₐ—was defined and considered, which represented the combined effects of the stress confinement and of tissue laser light absorption, both of which effects lead to increase of the ablation-caused stress of the target.

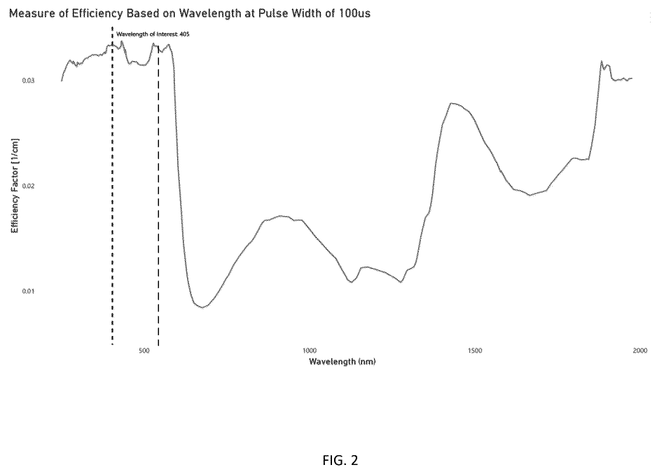

The spectral dependency of this figure of merit for pulsed laser irradiation (optionally—for a variety of operating pulse widths and/or duty cycles, but in a specific example—of about 100 microseconds) across the spectral region including both the visible and the near-infrared light was then assessed (see curve 200 of FIG. 2) to identify the desired spectral regions of operation of the laser diode source as the region within which such figure of merit is substantially maximized. The plot of FIG. 2 evidences that, based on available off-the-shelf high-power laser diodes, the choice of the laser diode source operating in the vicinity of 405 nm for example (indicated with one of the two vertical dashed lines) warrants the efficient exposure of the target tissue to the ablation stress. (Another possible choice of the spectral region of operation of the laser diode, derived in this particular example, is identified by the second vertical dashed line of FIG. 2) Table 1 provides values of various material characteristics used to assess the critical relaxation times of tₜ=0.0013 s and tₘ=4.20e−09s for the chosen in this example 405 nm laser irradiation of the target tissue.

TABLE 1
Material Characteristics and Values

| Property (Units) | Notation | Value |
|---|---|---|
| Density (kg/m³) | ρ | 1080 |
| Thermal | K | 0.55 |
| Conductivity | | |
| (W/(m*K)) | | |
| Specific Heat | Cₚ | 3500 |
| (J/(kg*K)) | | |
| Absorption | μₐ | 790.58 |
| Coefficient at | | |
| 405 nm (1/cm) | | |
| Reduced Scattering | μ′ₛ | 105.21 |
| Coefficient at | | |
| 405 nm (1/cm) | | |
| Penetration | δ | 0.068 |
| Depth (cm) | | |
| Thermal | κ | 1.46e−07 |
| Diffusivity (m²/s) | | |
| Speed of Sound (m/s) | v | 1633 |
| Thermal Critical | tₜ | 0.0013 |
| Relaxation Time (s) | | |
| Mechanical Critical | tₘ | 4.20e−09 |
| Relaxation Time (s) | | |

(sources: Nahirnyak, V. M., et al., in J. Acoust. Soc. Am. 119, 3766-3772, 2006; and Moritz Friebel, et al., in J. Biomed. Opt. 14, 034001, 2009).

### Some Details on Used Instrumentation

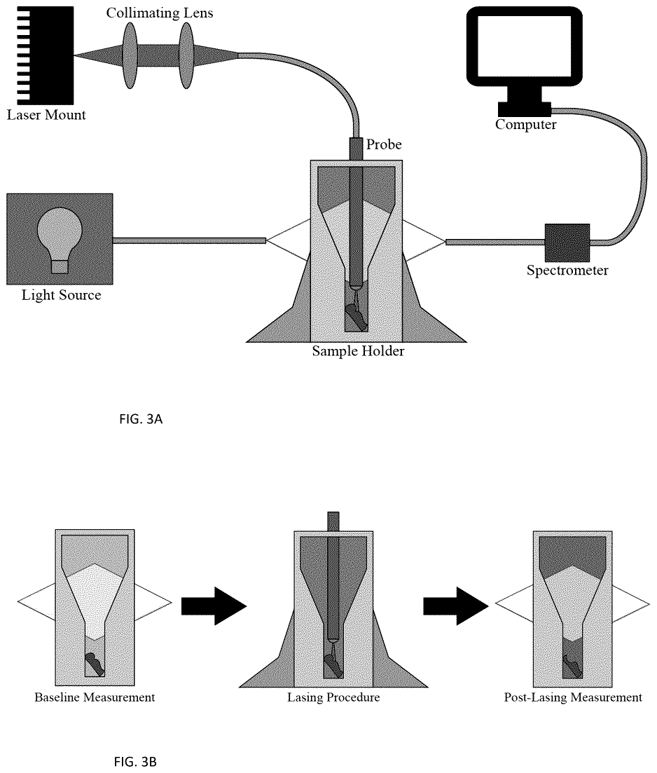

Some portions of the apparatus 100 of FIG. 1, schematically presented in FIGS. 3A, 3B as structured according to the idea of the invention, featured a 405 nm, 1000 mW laser diode (L405G1, Thorlabs, Newton, NJ) attached to a laser mount. The laser diode rested on the laser mount (LDM90, Thorlabs, Newton, NJ) and was connected to an external laser diode controller (ITC4020, Thorlabs, Newton, NJ). The purpose of the laser diode controller was to electronically govern the operation of the laser source to produce pulsed light output at a user-defined frequency and duty cycle. The optical output of the laser diode was collimated and coupled to a custom fiber optic probe (Fiberoptic Systems Inc., Simi Valley, CA) with an approximately 1 mm bundle borosilicate fiber core (0.55 NA) and a lens with a 1.5 mm diameter, 2 mm focal length, and 0.38 NA attached at the proximal end of the bundle. FIGS. 3A and 3B are benchtop test beds used to quantitatively measure the effect of laser ablation.

### Embodiments of a Thrombus Analog

The performance and effectiveness of the ablation process were determined using thrombus analogs, with various thrombus hardness and adhesive properties, fabricated with sheep's blood (Duffy, S. et al., in J. NeuroInterventional Surg. 9, 486, 2017) to analyze the effects of ablation. A sample of blood was processed through a centrifuge at 600g for 15 minutes. From the separated blood components, red blood cells and extracellular fluid (ECF) materials were distributed to sample containers. Calcium chloride, (2.04% by weight), was added to the blood sample at a 1:9 volume ratio (calcium chloride to blood sample). The blood sample was placed in a water bath at 37° Celsius for one hour for the blood to coagulate into a thrombus.

For one experiment, thrombus models of 20% ECF and 100% ECF were created, in order to test the nature of extreme cases of thrombus composition. By testing the 20% ECF, for example, one can observe the laser thrombolysis effects on a primarily red blood cell composed thrombus, which is seen as more brittle. Conversely, we the 100% ECF samples were tested due to their representation of the more complex case of thrombectomy (due to the tightly bound structure; Jackson, S. P., in Nat. Med. 17, 1423-1436, 2011).

### Experimental

A thrombus sample was placed into a cuvette that contained 1 mL of deionized water to validate the effectiveness of the thrombolysis instrument. The cuvette was first placed into a spectrometer (OceanOptics HR2000+, Dunedin, Florida) for recording a baseline intensity. The cuvette was then removed and put on a stage, where the terminal end of the laser output was positioned approximately 0.5 millimeters above the thrombus surface. The laser diode controller implemented the assigned pulse widths and duty cycles. The thrombus was irradiated with the laser light at the set pulse width for five minutes. The cuvette was removed from the stage and placed back into the spectrometer, where post-ablation intensity recordings were taken.

Ten samples were taken for each combination of ECF composition, pulse width, and duty cycle. (The pulse widths used were 500 μs and 1000 μs; the duty cycles used were 0.1, 0.2, and 0.5). A negative control was implemented, where the thrombus sample remained suspended within the cuvette media with no laser irradiation. A positive control was implemented as well, where the continuous-wave (CW) laser light was used to irradiate the thrombus sample (with no pulsing). The laser source output power was measured at about 500 mW while in continuous (CW) mode before beginning the pulsing operation.

### Data Analysis

The effectiveness of the implemented laser thrombolysis procedure was quantified by the extent to which thrombus/blood particles were dislodged from the thrombus. Specifically, the absorbance of the thrombus particulates was calculated from the spectrophotometer (see FIG. 3A) intensity measurements using Beer-Lambert's law. The intensity recordings were converted into absorbance in the 200 nm to 1100 nm wavelength range as according to:

$$Absorbance = log ( \frac{I}{{I}_{0}} )$$

with/as the post-experimental intensity reading and I₀ as the baseline intensity reading. The absorbance value at 405 nm (the identified in the specific example illumination wavelength) was used as a quantitative metric of laser thrombolysis efficacy. Note that the absorbance is inversely proportional to effectiveness, with lower values indicating greater laser effects. The material ablated from the thrombus disperses into the surrounding liquid. The results of the measurements of absorbance of this dispersed thrombus material represent the effectiveness of the ablation process. Finally, a t-test (known in the art to produce assessment of the difference between the means of exactly two groups) was performed to test if the used groups of samples were statistically different (p<0.05) from the group irradiated with the CW laser output (the Continuous control group and from that not irradiate with the laser output at all (the OFF control group).

### Results

(A) Results Produced with 100% ECF Analogs

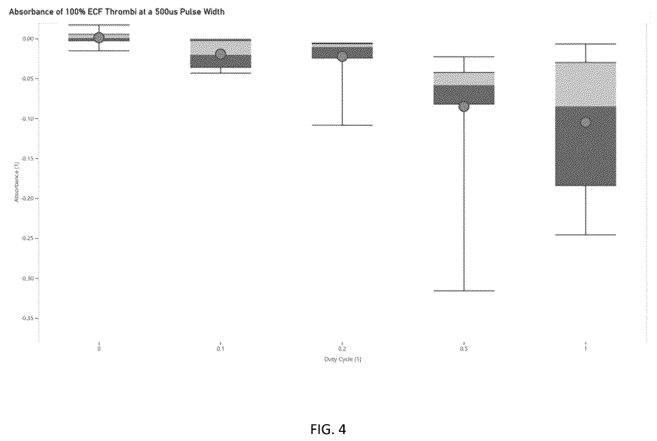

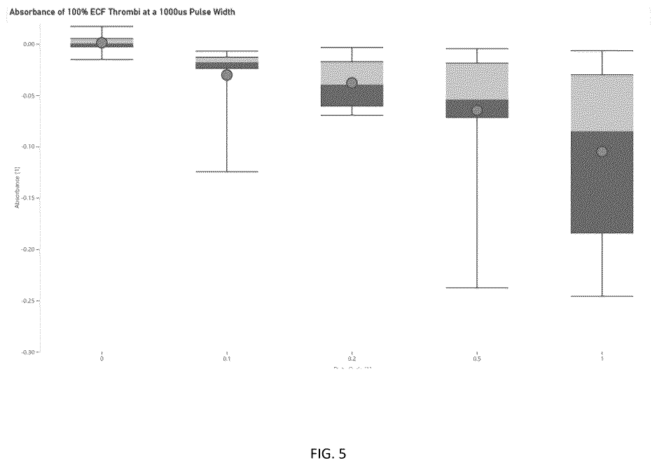

FIGS. 4 and 5 present plots of the absorbance within the fluid media in the cuvette for the respective duty cycles at pulse widths of 500 μs and 1000 μs, respectively, for thrombi with ECF at 100 vol % (i.e. fully fibrin). The ablative effect of the laser light on these samples evidenced a significantly different absorbance as compared to the negative control. However, the pulsed lasing did not perform better than in the case of the positive control. Further, there were no significant differences between the pulse widths used in these tests.

TABLE 2
Mean Absorbance of Surrounding Media with ECF 100 vol %

| | Pulse Width | |
|---|---|---|
| Duty Cycle | (us) 500 | 1000 |
| Continuous | −0.11 | −0.11 |
| OFF | 9.20E−04 | 9.20E−04 |
| 0.1 | −0.020*+ | −0.030* |
| 0.2 | −0.023+ | −0.038*+ |
| 0.5 | −0.085* | −0.065* |

*Indicates significantly different from OFF Control
+Indicates significantly different from Continuous Control

Results with 20% ECF Analogs

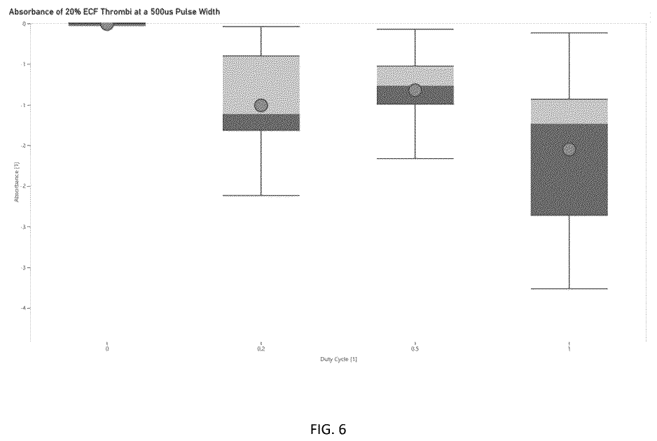

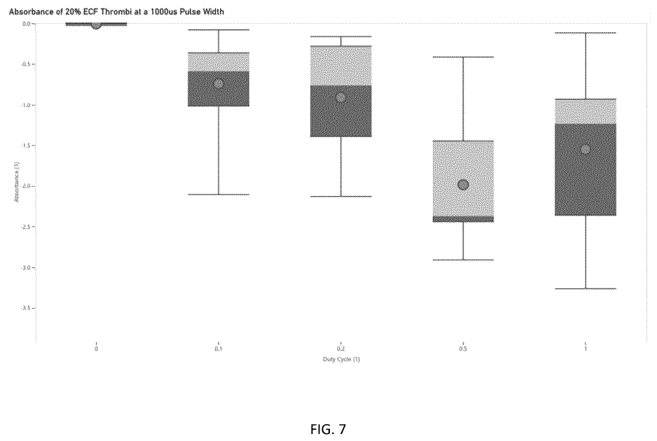

FIGS. 6 and 7 display plots of the absorbance within the fluid media in the cuvette for the respective duty cycles at laser output pulse widths of 500 μs and 1000 μs, respectively, for thrombi with ECF at 20 vol %. The ablative effect produced by the laser light output on these samples presented a significantly different absorbance as compared to the negative control. Similarly to the 100% ECF analogs, the pulsed lasing did not show a statistically significant difference from the positive control or from results produced by irradiation with the different pulse widths. However, a sharp increase in changes in absorbance as the duty cycle increases can be observed.

TABLE 3
Mean Absorbance of Surrounding Media with ECF 20 vol %

Pulse Width (us)

| Duty Cycle | 500 | 1000 |
|---|---|---|
| Continuous | −1.55 | −1.55 |
| OFF | −0.0046 | −0.0046 |
| 0.1 | NaN | −0.74*+ |
| 0.2 | −1.01* | −0.91*+ |
| 0.5 | −0.82* | −1.99* |

*Indicates significantly different from OFF Control
+Indicates significantly different from Continuous Control

### DISCUSSION

Implementation of successful laser-assisted aspiration thrombolysis does not require the use of complicated laser sources: multiple fibrin-heavy thrombi analogs, fabricated with various concentrations (including thrombi that contained nearly only fibrin) were successfully ablated with the use of a standard, off-the-shelf laser diode operating in a pulsed mode with greater than a nanosecond pulse duration. An important distinguishing, from the systems currently used in related art, factor is the absence of excimer and crystal-based complex laser systems and pulses of short durations (in a sub-nanosecond range), sometimes requiring the use of femtosecond laser systems) while employing simple diode lasers having a wider diversity of spectral and power options, rather than conventional systems that use crystal lasers. The ability to use longer pulses of assisting laser radiation render practical concerns-such as pulse-dispersion in the light delivering optical fiber-substantially irrelevant, thereby providing an additional degree of operational freedom of using a substantially broader, more divers (as compared with related art) optical fibers (for example, optical fibers that are not judiciously structured to compensate for pulse dispersion).

The implications of utilizing a low-cost approach to laser thrombolysis within various clinical settings demonstrate that, with the current off-the-shelf solutions, laser thrombolysis is feasible without the use of expensive crystal-based lasers. Furthermore, as seen in the ASTER trial (see, for example, Lapergue, B. et al., in JAMA 318, 443-452, 2017)—and while the composition of the thrombi does impact the efficiency of the surgery—the laser thrombolysis procedure can be used at least as assistance in removing more difficult thrombi.

Whether or not explicitly discussed above and/or indicted in the drawings, the skilled person will readily appreciate that embodiment of methodology of the invention may require the use of a processor (and/or dedicated electronic circuitry) controlled by instructions stored in a memory. The memory may be random access memory (RAM), read-only memory (ROM), flash memory or any other memory, or combination thereof, suitable for storing control software or other instructions and data. Those skilled in the art should also readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. In one specific case, the terms “approximately”, “substantially”, and “about”, when used in reference to a numerical value, represent a range of plus or minus 20% with respect to the specified value, more preferably plus or minus 10%, even more preferably plus or minus 5%, most preferably plus or minus 2% with respect to the specified value. As a non-limiting example, two values being “substantially equal” to one another implies that the difference between the two values may be within the range of +/−20% of the value itself, preferably within the +/−10% range of the value itself, more preferably within the range of +/−5% of the value itself, and even more preferably within the range of +/−2% or less of the value itself. The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes.

The term “element A and/or element B” is defined to cover each and every of “element A”, “element B”, “element A and element B”.

For the purposes of this disclosure and accompanying claims, a real-time performance of a system is understood as performance which is subject to operational deadlines from a given event to a system's response to that event. For example, a real-time acquisition of information about the target (such as, for example, information about material characteristics of the target-which may affect the regime of operation of an embodiment of the apparatus) may be one triggered by the user and executed substantially simultaneously with and without interruption of preparation of the apparatus to removal of the target.

Disclosed aspects, or portions of these aspects, may be combined in ways not listed above. Accordingly, the invention should not be viewed as being limited to the disclosed embodiment(s). For example, the embodiment of the apparatus of the invention can be additionally equipped with an optical-waveguide based channel configured to assist in determination of the material composition of the target-whether in real time with performing at least one step of the operation of the apparatus or ahead of such operation.

What is claimed is:

1. An apparatus for target removal, the apparatus comprising:
an aspiration system including a tubular body and a piping network operably cooperated with the tubular body, the aspiration system configured to generate suction at an end of the tubular body;
an optical system containing a laser that is configured to operate in a pulsed regime to generate pulses of laser radiation having durations substantially longer than a nanosecond at a wavelength that is defined at least in part by a spectral dependency of a figure of merit (FOM) that represents both a stress confinement within a material of the target, and absorption of the laser radiation by the material; and
an optical waveguide disposed to deliver the laser radiation to the end of the tubular body.

2. An apparatus according to claim 1, wherein the laser is a laser diode device.

3. An apparatus according to claim 1, further comprising:
electronic circuitry electrically coupled with the aspiration system and/or the optical system and configured:
to govern an operation of an aspiration system driver to reversibly produce the suction and to operate the optical system to reversibly produce said pulses of laser radiation; and/or
to vary a pulse width, a pulse duty cycle, and an average power of the laser radiation; and/or
to vary a strength of the suction produced by the aspiration system.

4. An apparatus according to claim 1, further comprising a sheath dimensioned to slidingly fit into a blood vessel and to removably fit therein both the tubular body and the optical fiber.

5. An apparatus according to claim 1, wherein the tubular body is dimensioned to removably fit the optical waveguide in the hollow thereof.

6. A method for removal of a target, the method comprising:
using the apparatus of claim 1:
determining the spectral dependency of the FOM within a pre-defined spectral range based at least on pre-assessed material properties of the target;
identifying the wavelength of operation of the laser in the apparatus based at least on the spectral dependency of the FOM;
operating the laser to generate pulses of the laser radiation substantially at the wavelength of operation; and
substantially simultaneously with or in alternating fashion with said operating the laser, generating the suction at the end of the tubular body with the aspiration system.

7. A method according to claim 6, further comprising:
channeling the laser radiation within the optical waveguide and spatially converging, outside of the tubular body, the laser radiation delivered along the tubular body to the end of the tubular body.

8. A method according to claim 6, comprising:
positioning the end of the tubular body and an output end of the optical waveguide in a vicinity of the target at a reference surface,
applying the laser radiation to the target to dislodge the target with respect to the reference surface and/or to chip away a portion of the target from a body thereof, and
repositioning the target with respect to the reference surface and/or the portion of the target with respect to the body of the target with the suction.

9. A method according to claim 6, wherein said identifying the wavelength includes determining a value of a wavelength-dependent product of a stress confinement factor in the material of the target and a value of a wavelength-dependent coefficient of absorption of laser radiation by the material of the target.

10. A method according to claim 6, wherein the target includes a biological tissue affixed inside a bodily cavity.

11. A method according to claim 6, wherein the target includes a thrombus within a blood vessel.

12. A method according to claim 6, further comprising:
varying a pulse width, a pulse duty cycle, and/or an average optical power of the laser; and
varying a strength of the suction produced by the aspiration system.

13. A method according to claim 12, determining one or more of the pulse width, the duty cycle, the average power, and the strength of aspiration suction are at least in part based on a material composition of the target.

14. A computer program product for use on a computer system operating an aspiration thrombectomy apparatus, the computer program product comprising a computer usable tangible non-transitory storage medium having computer readable program code thereon, the computer readable program code including program code for one or more of:
governing an operation of a laser diode to generate laser radiation at a wavelength within the range in a pulsed regime with a pulse duration substantially longer than a nanosecond;
switching off and on an aspiration system of the apparatus to reversibly generate suction at an identified end of a suction catheter of the aspiration system to which the laser radiation has been delivered;
varying a pulse width, a duty cycle, and/or an average power of the laser radiation; and
varying a strength of the suction generated by the apparatus,
wherein the operating the aspiration thrombectomy apparatus includes producing the suction substantially simultaneously with generating the laser radiation or producing the suction in alternating fashion with the generating the laser radiation.
